(19) United States
(12) Patent Application Publication
(10) Pub. No.: US 2025/0034674 A1
(43) Pub. Date: Jan. 30, 2025

(54) Method for manufacturing an annealed steel sheet

(71) Applicant: ArcelorMittal, Luxembourg (LU)

(72) Inventor: Thomas DIEUDONNE, Semécourt (FR)

(21) Appl. No.: 18/716,994

(22) PCT Filed: Dec. 6, 2022
(86) PCT No.: PCT/IB2022/061805

§ 371 Date: Jun. 6, 2024

(30) Foreign Application Priority Data
Dec. 17, 2021 (WO) PCT/IB2021/061906

(51) Int. Cl. C21D 11/00; C21D 1/26; C21D 9/46

(52) U.S. Cl. CPC C21D 11/00; C21D 1/26; C21D 9/46

(57) ABSTRACT

A method for manufacturing a heat-treated stool sheet, submitted to an annealing process following a temperature curve T as a function of time t in a furnace having atmosphere including hydrogen, in which the hydrogen content targeted in the steel sheet at the end of a step of the annealing process is defined.

The present invention relates to a method for manufacturing an annealed steel sheet in which the process parameters are selected to obtain a defined hydrogen value at the end of any steps of the annealing process.

### BACKGROUND

A steel sheet is made of grains in which atoms are arranged in crystal lattice, thus forming structure of the steel. Spaces between these atoms are called interstitial sites. The arrangement of atoms is not totally regular, and some arrangement defect can occur, which is the case for dislocations which are linear defect.

During the heat treatment of a steel sheet, hydrogen atoms present in the atmosphere of the furnace, can easily penetrate the steel and can be absorbed. Indeed, hydrogen can diffuse into the crystal lattice due to its atomic size of the same order of magnitude as the size of the interstitial sites of the crystal lattice. Hydrogen atoms may progressively diffuse and be trapped inside the defects such as dislocations.

The introduction and diffusion of hydrogen in the steel sheet is one of the mechanism responsible of the brittleness of the steel sheet, which could lead for example, to cracks formation along grain boundaries and/or dislocations gliding planes.

Because of hydrogen, steel strip can suffer ductility lost, also called hydrogen embrittlement.

### SUMMARY OF THE INVENTION

It is an object of the present invention to provide a method for manufacturing an annealed steel sheet in which the process parameters are selected to obtain a defined hydrogen value at the end of any steps of the annealing process.

The present invention provides a method for manufacturing an annealed steel sheet having a chemical composition, submitted to an annealing process following a thermal path, in which the temperature can be measured by sensors in a furnace having atmosphere comprising hydrogen, said steel sheet comprising grains in which atoms are arranged in a crystal lattice, thus forming the microstructure of the steel, including dislocations and interstitial sites, said method comprising the following successive steps:
defining the hydrogen content C_{total-targeted} targeted in the steel sheet at any step of the annealing process
defining at least two temperature curves Tₙ of the annealing process as a function of time t, n being the number of curves
defining the amount of hydrogen in the atmosphere of the furnace during the annealing process
determining the microstructure of the steel sheet as a function of the thermal path,
computing the solubility of the hydrogen C_{H}, at the surface of the steel sheet as a function of the microstructure, the temperature Tₙ and hydrogen partial pressure p_{H2},
computing the volume concentration of trapped hydrogen in dislocations C_{T} and the volume concentration of hydrogen in interstitial sites C_{L}, as a function of temperature Tₙ, of trapping rate of hydrogen k and of detrapping rate of hydrogen p, of C_{H}, and of the microstructure,
Calculating the hydrogen content Cₜₒₜₐₗ=C_{L}+C_{T} at any time of the annealing process
optionally outputting through a computer display the hydrogen content Cₜₒₜₐₗ at any time to a user.
Selecting the temperature curves Tₙ and the composition atmosphere leading to Cₜₒₜₐₗ as close as possible to C_{total-targeted}
providing the steel sheet with the said chemical composition,
annealing the steel sheet according to the said selected temperature curve T=Tₙ, as a function of time t, in the said selected composition atmosphere.

Hereinafter, C_{L} designates the concentration of hydrogen in the interstitial sites of the crystal lattice of the steel sheet and C_{T} the concentration of trapped hydrogen in the steel sheet. Dislocations are the only trapping sites considered in the invention, homogeneously distributed in the microstructure.

### BRIEF DESCRIPTION OF THE DRAWINGS

The invention will now be described in detail and illustrated by examples without introducing limitations, with reference to the appended figures:

FIG. 1 represents the temperature curve T0 and T1,

FIG. 2 illustrates the cells used in one embodiment of the method of the invention, to represent the microstructure at point F1 of temperature curve T1

FIG. 3 represents the time evolution of C_{L}, C_{T} and Cₜₒₜₐₗ=C_{L}+C_{T} of trial 1

FIG. 4 represents the time evolution of C_{L}, C_{T} and Cₜₒₜₐₗ of trial 2

FIG. 5 represents the time evolution of C_{L}, C_{T} and Cₜₒₜₐₗ of trial 3

FIG. 6 represents the time evolution of C_{L}, C_{T} and Cₜₒₜₐₗ of trial 4

FIG. 7 represents the time evolution of C_{L}, C_{T} and Cₜₒₜₐₗ of trial 5

FIG. 8 represents the time evolution of C_{L}, C_{T} and Cₜₒₜₐₗ of trial 6

### DETAILED DESCRIPTION

A method for manufacturing an annealed steel sheet in which the process parameters are selected to obtain a defined hydrogen value at the end of any steps of the annealing process is provided, said method comprising the following successive steps:

The first step of the method according to the invention is to define the hydrogen content C_{total-targeted} targeted in the steel sheet at the end of a step of the annealing process.

During this annealing, the sheet is subjected to at least one heating step and one cooling step, according to a thermal path. Usually, heat treatments can be performed in an oxidizing atmosphere, i.e. an atmosphere comprising an oxidizing gas being for example: O₂, CH₄, CO₂ or CO. They also can be performed in a neutral atmosphere, i.e. an atmosphere comprising a neutral gas being for example: N₂, Ar or He. Finally, they also can be performed in a reducing atmosphere, i.e. an atmosphere comprising a reducing gas being for example: H2 or HNx.

In a preferred embodiment, the thermal path can also include at least one isothermal holding step, that can usually be preceded by a heating step and followed by a cooling step. The cooling step can comprise an isothermal holding, called an overaging sub-step followed by a subsequent cooling step. A hot-dip coating step in a hot metal bath can also be used during such thermal path and is another type of isothermal holding as the metallic sheet dipped in such hot metal bath will be maintained at the bath temperature during its retention time in such bath.

Said annealing can be, for example, recrystallization annealing, recovery or a tempering, and can be followed by these heat-treatments:
quench and tempering,
quench and partitioning.

The thermal path according to the invention corresponds to the successive temperatures T, heating and cooling rates and time spent in each section of the annealing process and optional subsequent heat-treatments.

The temperatures of the thermal path can be measured by sensors during the annealing and optional subsequent heat-treatments, or through calculations done with the use of a software.

Then at least two temperature curves Tₙ of the annealing step as a function of time t, n being the number of curves, in a furnace having atmosphere with hydrogen is defined. The amount of hydrogen in atmosphere is defined. The atmosphere in the furnace can comprise H₂, N₂ and O₂.

The next step of the method according to the invention is to determine the microstructure of the steel sheet as a function of the thermal path of the annealing process, through for example calculations done with the use of a software like COMSOL®. The microstructure can also be determined thanks to sensors in furnace able to measure the austenite content in the steel, like X-CAP®.

In the frame of the present invention, the evolution of the microstructure occurs instantaneously only at certain points, corresponding to a phase change at given temperatures.

The solubility of hydrogen C_{H} at the surface of the steel sheet is then calculated. The solubility of hydrogen is the aptitude of hydrogen to be dissolved in the steel sheet. This solubility depends on the temperature, partial pressure of hydrogen and on the phases present in the microstructure of the steel sheet. It can be calculated through the following equations [1] and [2] that will be described.

For a temperature Tₙ below or equal to Ac3, preferably from 280K to 1184K:

$$log ( {C}_{H} ) = 0.5 log ( {p}_{{H}_{2}} ) - 3 - \frac{1500}{{T}_{n}} \qquad [ 1 ]$$

In the first part of the temperature curve where heating takes place, ferrite is the main structure in the steel sheet. The solubility of hydrogen C_{H} in ferrite is adequately calculated using above formula [1].

In the last part of the temperature curve where cooling takes place, part of the austenite formed above Ac3 can transform in bainite and/or martensite, depending on the composition of the steel and on the cooling rate. In that part of the curve, the solubility of hydrogen C_{H} in bainite and martensite is the same as in ferrite and can be obtained adequately using also above formula [1].

For a temperature Tₙ above Ac3, preferably from 1184K to 1667K:

$$log ( {C}_{H} ) = 0.5 log ( {p}_{{H}_{2}} ) - 2.9 - \frac{1490}{{T}_{n}} \qquad [ 2 ]$$

In the middle part of the temperature curve where holding at high temperature can take place, austenite is the main phase in the steel sheet, and the solubility of hydrogen C_{H} in austenite is adequately calculated using formula [2] above.

In both equations [1] and [2], C_{H} is expressed in at % and p_{H2} is the hydrogen partial pressure in the furnace, expressed in Pa. These both equations are from the publication of Fromm & Jehn “Hydrogen in Elements”, (Bull. Alloys Phase Diagrams, 5 (3), 323-326 (1984)).

Determining the solubility of hydrogen C_{H} at the surface of the steel sheet is required as an input for the next step of the method according to the invention, wherein the volume concentration of hydrogen in the interstitial sites of the crystal lattice C_{L} and the volume concentration of trapped hydrogen C_{T} in the steel sheet, both expressed in mole of hydrogen by m³ of iron (mol_{H}/m³_{Fe}), are computed with numerical simulations through the resolution of the following equations:

$$\frac{\partial {C}_{T}}{\partial t} = \frac{k}{{N}_{L}} {C}_{L} ( {N}_{T} - {N}_{A} \times {C}_{T} ) \qquad [ 3 ]$$

$$\frac{\partial {C}_{L}}{\partial t} + \frac{\partial {C}_{T}}{\partial t} \text{?} \qquad [ 4 ]$$

$$\text{?} \text{indicates text missing or illegible when filed}$$

The different parameters and constants of both equations [3] and [4] will now be explained.

D_{L} is the diffusion coefficient in the crystal lattice, expressed in m²/s, which depends on the temperature and phases present in the steel sheet at that temperature. This diffusion coefficient expresses the aptitude of hydrogen to diffuse inside a material. The higher the coefficient, the more easily the hydrogen diffuses.

In ferrite, martensite and bainite this coefficient diffusion D_{L} of hydrogen is the same, and is calculated through the following equation:

$${D}_{L} = 5.12 * {10}^{- 4} {e}^{- \frac{4150}{{RT}_{n}}} \qquad [ 5 ]$$

In austenite, the coefficient diffusion D_{L} of hydrogen is calculated according to the following equation:

$${D}_{L} = 5.8 * {10}^{- 3} {e}^{- \frac{53800}{{RT}_{n}}} \qquad [ 6 ]$$

R=8.314 J/mol·K being the universal gas constant and Tₙ the temperature expressed in K.

These equations show that at equivalent temperature, the hydrogen diffuses more easily in the ferrite, martensite and bainite than in austenite.

N_{L}=5.2×10²⁹ sites/m³ is the volume density of the interstitial sites in the steel sheet. In the method according to the invention, N_{L} is the same in all the phases of microstructure.

N_{T} is the volume density of dislocations, expressed in sites/m³. Dislocations have an associated trapping energy of E_{B}=27000 J/mol. In the method according to the invention, one dislocation can trap one or more hydrogen atoms. The volume density of dislocations N_{T} is calculated by using the surface density of dislocation ρ_{dis} expressed in sites/m², thanks to the following formula:

$${N}_{T} = α \frac{{ρ}_{dis}}{{a}_{bcc} \frac{\sqrt{3}}{2}} \qquad [ 7 ]$$

with α being the number of dislocations per Burger's vector, which represents the ability of dislocations to trap H atoms. The higher this coefficient, the more the dislocations trap the hydrogen atoms. a_{bcc} is the lattice parameter in the bcc structure expressed in angstroms. In the frame of the invention, this lattice parameter is the same in ferrite, martensite and bainite which are all bcc structures. In a preferred embodiment, those parameters can take the following values:
α=7
a_{bcc}=2.87 Å

In the frame of the invention atoms of hydrogen cannot be trapped by dislocations in austenite, because of their low diffusion coefficient.

k and p are respectively the hydrogen trapping and detrapping rates, expressed in s⁻¹, corresponding to the quantity of hydrogen atoms respectively trapped and detrapped, as a function of time, defined by the following equations

$$k = {k}_{0} exp ( \frac{- {E}_{T}}{{RT}_{n}} ) \qquad [ 8 ]$$

$$p = {p}_{0} exp ( \frac{- {E}_{D}}{{RT}_{n}} ) \qquad [ 9 ]$$

E_{T}=4 150 J/mol, being the energy of trapping, which is the energy that hydrogen atom must provide to be trapped, and E_{D}=E_{T}+E_{B}=31150 J/mol being the energy that hydrogen must provide to be detrapped.

Constants k0 and p0 are the hydrogen trapping and detrapping coefficient expressed in s⁻¹. They are used as fitting parameters for the calculation of C_{T} and C_{L} together with N_{T} in the different phases of the microstructure. Such fitting parameters can be determined through a comparison between experiments performed on a given steel composition and calculations according to the invention, iterated until experimental and calculated values converge.

Finally, N_{A}=6.02×1023 mol⁻¹ is the Avogadro number.

As described above, some of the calculations performed in the frame of method of the invention depend on the phases present in the steel at a given point of the temperature curve. Moreover, equation [3] depend on the depth x of the steel portion for which the calculations are done. To give an accurate evaluation of the hydrogen trapped in the entire thickness of the steel sheet, it is preferred to consider that the sheet is made of the repetition of N cells of 5 μm×5 μm, in order to simulate at least part of the thickness of the sheet. In a preferred embodiment, half—of the thickness of the sheet is used, N being calculated through the formula:

$$N = thickness of the steel sheet / ( {2}^{*} 5 μm )$$

The other half thickness of the steel sheet behaves exactly like the first one and that the diffusion of hydrogen is homogeneous in the full length of the sheet.

C_{H} values can then be calculated using equations [1] and [2] all along the temperature curve. Such C_{H} values are then used as the C_{L} values for the first row of cells.

Formula [3] and [4] can be successively applied to each cell to finally provide the values of C_{T} and C_{L} for the full thickness of the sheet.

In the last step of the method according to the invention, the total hydrogen content Cₜₒₜₐₗ is determined by calculating the sum of C_{L} and C_{T} at any time, before to be optionally output to a user.

The temperature curve Tₙ and the composition atmosphere leading to Cₜₒₜₐₗ as close as possible to C_{total-targeted} is selected, in order to further produce an annealed steel sheet according to a temperature curve T=Tₙ, having a hydrogen content the closest of C_{total-targeted}.

The invention will be now illustrated by the following example, which are by no way limitative.

### EXAMPLE

Cold rolled steel sheets having a composition consisting of 0.07% wt of C, 2.62% wt of Mn, 0.25% wt of Si, 0.3% wt of Cr, 0.16% wt of Al, 0.091% wt of Mo, the remainder of the composition being iron and unavoidable impurities resulting from the smelting, and a thickness of 1 mm, are supplied.

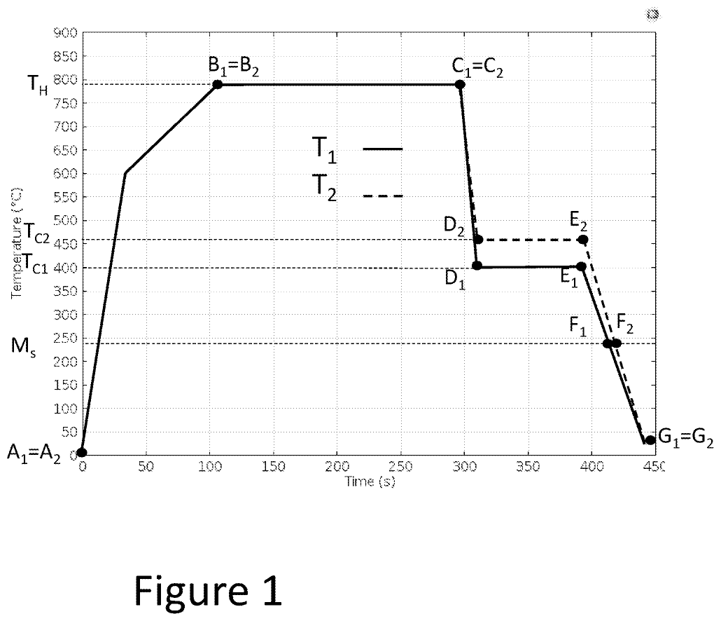

Such sheets can then undergo one annealing process. The temperature curves Tₙ (with n=2) as a function of time are described in FIG. 1. The sheets are heated to a temperature T_{H}, maintained at said temperature for a holding time t_{H}, in an atmosphere A_{H}, cooled to a cooling temperature T_{C} and maintained at said temperature T_{C} for a holding time t_{c} in an atmosphere Ac (hereinafter, this step is the overaging step), before to be cooled to room temperature (RT).

The temperature Ms of this grade is obtained by dilatometry measurement: Ms=240° C.

The lowest possible hydrogen value C_{total-targeted}=0 ppm value is sought at the end of the overaging step, in order to facilitate a subsequent zinc coating step.

The process parameters are gathered in Table 1:

TABLE 1
Process parameters

| | | Annealing | | | Cooling | | | | |
|---|---|---|---|---|---|---|---|---|---|
| | | | T_{H} | t_{H} | Cooling rate | T_{C} | | t_{C} | P_{H2} |
| Trials | Process | A_{H} | (° C.) | (s) | (° C./s) | (° C.) | A_{C} | (s) | (Pa) |
| 1 | T1 | 5% H₂, | 790 | 192 | 40 | T_{C1} = | 5% H₂, | 86 | 5066.5 |
| | | 95% N₂ | | | | 400 | 95% N₂ | | |
| 2 | T1 | 5% H₂, | 790 | 192 | 40 | T_{C1} = | 3% H₂, | 86 | 3039.9 |
| | | 95% N₂ | | | | 400 | 97% N₂ | | |
| 3 | T1 | 5% H₂, | 790 | 192 | 40 | T_{C1} = | 1% H₂, | 86 | 1013.3 |
| | | 95% N₂ | | | | 400 | 99% N₂ | | |
| 4 | T2 | 5% H₂, | 790 | 192 | 40 | T_{C2} = | 5% H₂, | 86 | 5066.5 |
| | | 95% N₂ | | | | 465 | 95% N₂ | | |
| 5 | T2 | 5% H₂, | 790 | 192 | 40 | T_{C2} = | 3% H₂, | 86 | 3039.9 |
| | | 95% N₂ | | | | 465 | 97% N₂ | | |
| 6 | T2 | 5% H₂, | 790 | 192 | 40 | T_{C2} = | 1% H2, | 86 | 1013.3 |
| | | 95% N₂ | | | | 465 | 99% N₂ | | |

### Hydrogen Content Evaluated According to the Invention

The microstructure of the steel sheet is estimated at each point (A1, B1, C1, D1, E1, F1, G1; A2, B2, C2, D2, E2, F2, G2) of the temperature curves as represented in FIG. 1.

The phase transformations of the microstructure occur instantaneously at the indicated points only. The estimated microstructures are gathered in Table 2:

TABLE 2
Estimated microstructures

| | | | | | Bainite |
|---|---|---|---|---|---|
| Process | Points | Ferrite(%) | Austenite(%) | Martensite(%) | (%) |
| T1 | A₁ | 100 | — | — | — |
| | B₁ | 30 | 70 | — | — |
| | C₁ | 30 | 45 | — | 25 |
| | D₁ | 30 | 25 | — | 45 |
| | E₁ | 30 | 25 | — | 45 |
| | F₁ | 30 | — | 25 | 45 |
| | G₁ | 30 | — | 25 | 45 |
| T2 | A₂ | 100 | — | — | — |
| | B₂ | 30 | 70 | — | — |
| | C₂ | 30 | 45 | — | 25 |
| | D₂ | 30 | 25 | — | 45 |
| | E₂ | 30 | 25 | — | 45 |
| | F₂ | 30 | — | 25 | 45 |
| | G₂ | 30 | — | 25 | 45 |

During the heating step up to T_{H}, ferrite is the main phase until temperature reaches AC1, wherein ferrite starts being transformed into austenite. During the holding step at T_{H}, respectively starting at points B1 and B2, the microstructure is then made of ferrite and austenite.

During the cooling starting at points C1 and C2, a part of austenite is transformed into bainite. Austenite continues to be transformed into bainite during the subsequent holding step, starting at D1 and D2. The microstructure during the cooling starting at E1 is the same as in the step between D1 and E1. The microstructure during the cooling starting at E2 is the same as in the step between D2 and E2.

The austenite is finally transformed into martensite at point F1 and F2, corresponding to the Ms temperature.

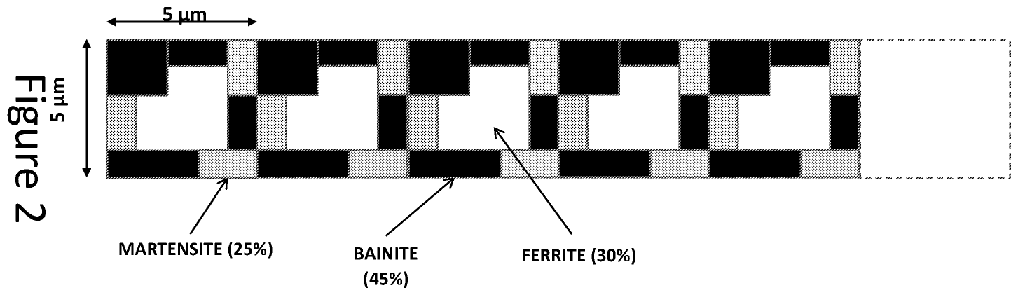

Half of the thickness of the steel sheet is simulated through the repetition of N=100 cells of 5 μm×5 μm. The phase percentages of table 2 are taken into account through the percentages of the surface of the cells, as illustrated on FIG. 2 corresponding to microstructure at point F₁: 25% of the surface of the cells represents the 25% of martensite inside the steel, 45% of the surface of the cells represents the 45% of bainite and 30% of the surface of the cells represents the 30% of ferrite inside the steel sheet.

C_{H} values are then calculated using equations [1] and [2]. Such C_{H} values are then used as the C_{L} values for the first row of cells.

N_{T} and the trapping and detrapping coefficients were fitted, using the following protocol. Steel sheets having a composition according to example 1, have been heated at a temperature of 850° C. in a furnace having an atmosphere consisting of 5% of H₂, the rest being N₂, and maintained at said temperature for a holding time of 260s, before being quenched. The experimental hydrogen content in each sheet has then been measured through TDA experiments, by heating the steel sheet at a heating rate of 1200° C./h.

After several iterations, the best fitting parameters have been chosen as follows:
k₀=10⁵ s⁻¹
p₀=10² s⁻¹
N_{T} in ferrite=10²⁴ sites/m³
N_{T} in bainite=5 10²⁴ site/m³
Nr in martensite=10²⁵ sites/m³

The volume concentration of hydrogen in the interstitial sites of the crystal lattice C_{L} and the volume concentration of trapped hydrogen C_{T} in the steel sheet are then computed through the resolution of equations [3] and [4], taking into account the microstructures described above, and for each of the point where a phase transformation occurs all along the thermal curves.

Cₜₒₜₐₗ (Cₜₒₜₐₗ=C_{L}+C_{L}) is then calculated at each of these points and optionally transmitted to an operator.

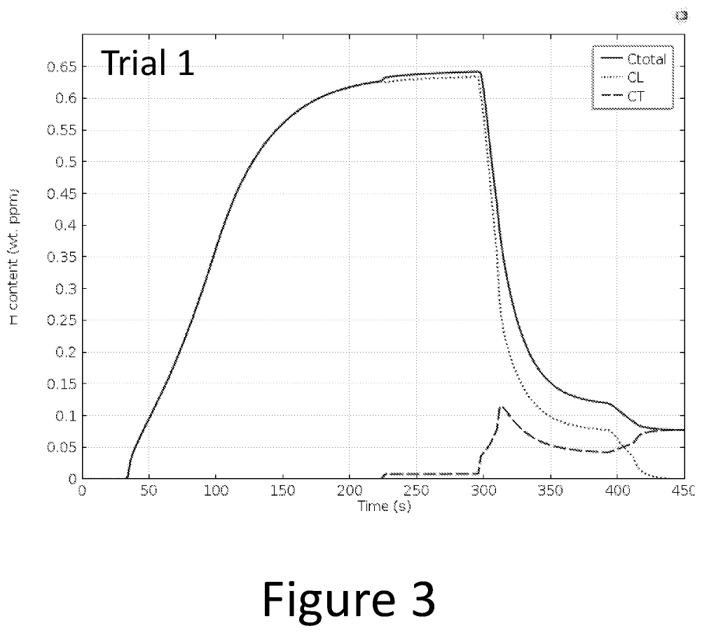

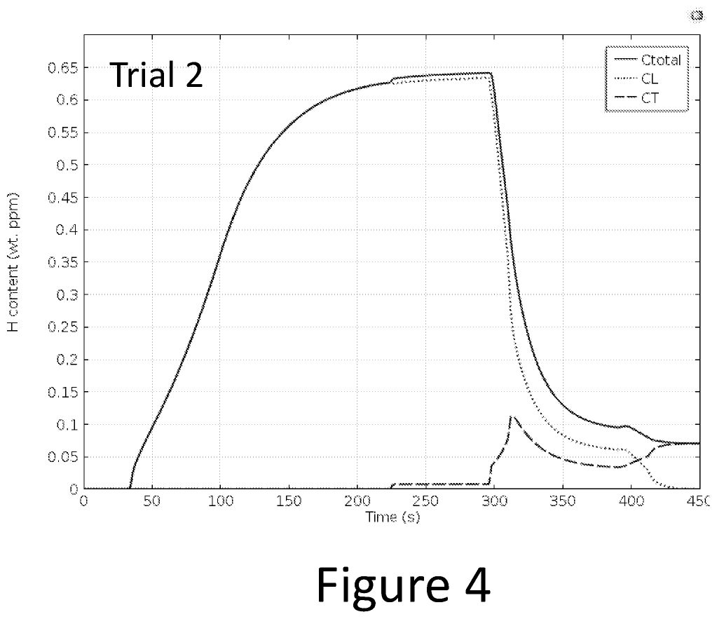

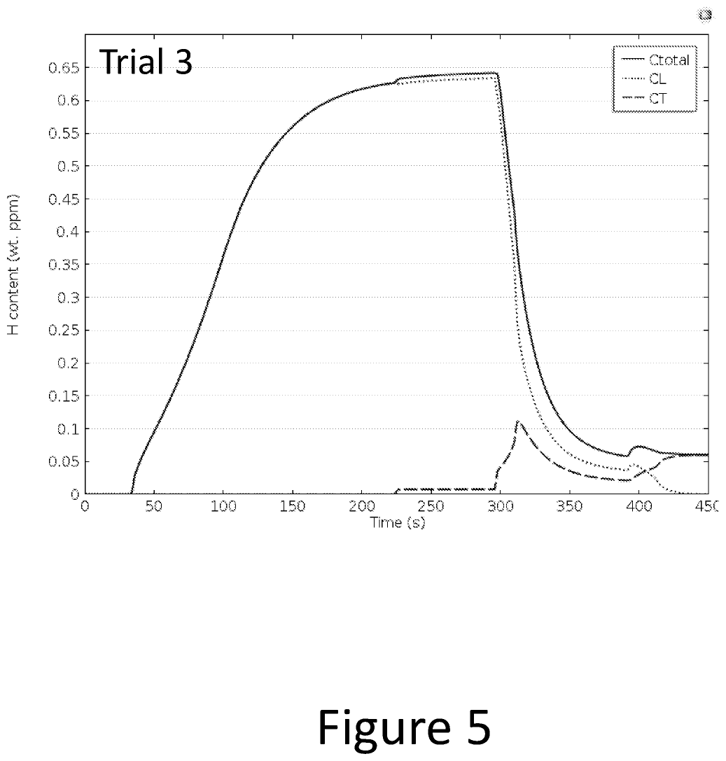

FIGS. 3, 4 and 5 represents respectively the evolution as a function of time of C_{L}, C_{T} and of the total hydrogen content Cₜₒₜₐₗ for trials 1, 2 and 3.

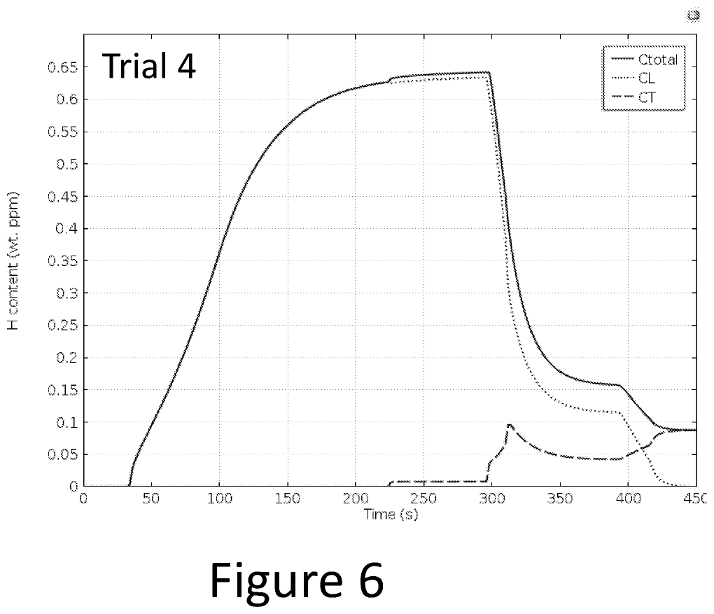

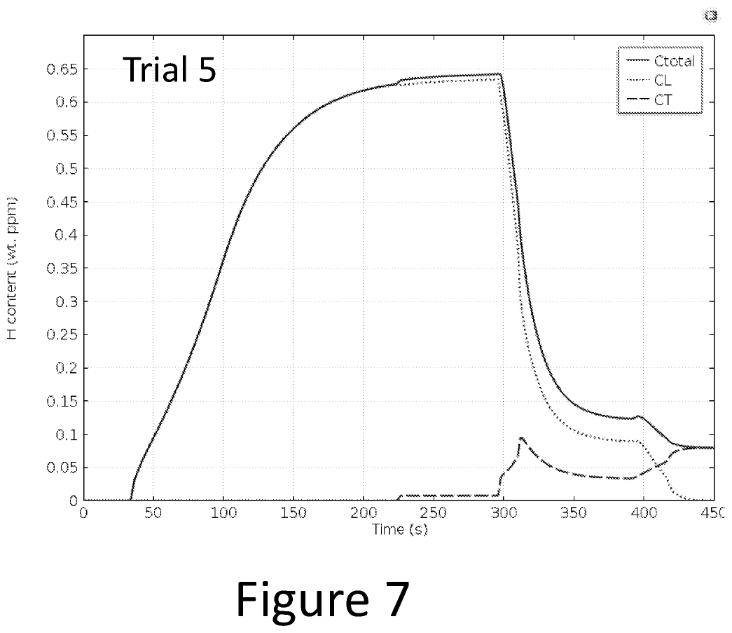

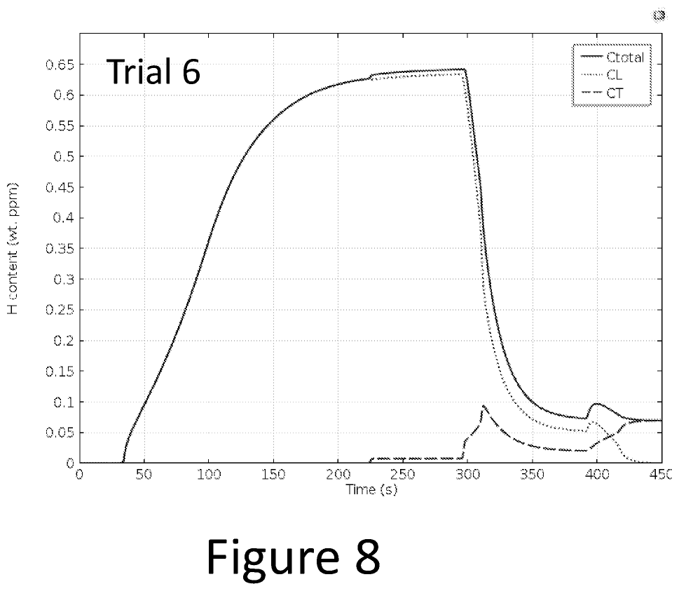

FIGS. 6, 7 and 8 represents respectively the evolution as a function of time of C_{L}, C_{T} and Cₜₒₜₐₗ for trials 4, 5 and 6.

The hydrogen contents at the end of overaging step (at point E₁ and E₂ on FIG. 1, corresponding to t=395 s) as determined by the method according to the invention are gathered in the following table:

| Trials | Cₜₒₜₐₗ (ppm) |
|---|---|
| 1 | 0.12 |
| 2 | 0.10 |
| 3 | 0.06 |
| 4 | 0.16 |
| 5 | 0.13 |
| 6 | 0.08 |

The method according to the invention evaluates that the amount of hydrogen in the steel sheet at the end of the overaging step can be reduced by reducing the hydrogen content in atmosphere during this overaging step, as it is seen in trials 1 to 3 wherein the hydrogen content in trial 3 performed with 1% of H2 during overaging step is lower than in trial 1 performed with 5% of H2. The same for trials 4 to 6, wherein the hydrogen content in trial 6 performed with 1% of H2 during overaging step is lower than in trial 4 performed with 5% of H2.

Moreover, the method according to the invention predicts that maintaining the steel sheet at a low cooling temperature, as performed in trials 1 to 3 with a T_{C}=400° C., reduces the amount of hydrogen compare to a higher temperature of trials 4 to 6.

1-7. (canceled)

8. A method for manufacturing an annealed steel sheet having a chemical composition, submitted to an annealing process following a thermal path, the temperature measureable by sensors in a furnace having atmosphere including hydrogen, the steel sheet including grains in which atoms are arranged in a crystal lattice, thus forming a microstructure of the steel, including dislocations and interstitial sites, the method comprising the following successive steps:
defining a hydrogen content C_{total-targeted} targeted in the steel sheet at any step of the annealing process
defining at least two temperature curves Tₙ of the annealing process as a function of time t, n being the number of curves
defining an amount of hydrogen in the atmosphere of the furnace during the annealing process
determining the microstructure of the steel sheet as a function of the thermal path,
computing a solubility of the hydrogen C_{H}, at the surface of the steel sheet as a function of the microstructure, the temperature Tₙ and hydrogen partial pressure p_{H2},
computing the volume concentration of trapped hydrogen in dislocations C_{T} and the volume concentration of hydrogen in interstitial sites C_{L}, as a function of the temperature Tₙ, of trapping rate of hydrogen k and of detrapping rate of hydrogen p, of C_{H}, and of the microstructure,
calculating a hydrogen content Cₜₒₜₐₗ=C_{L}+C_{T} at any time of the annealing process
selecting the temperature curves Tₙ and the composition atmosphere leading to Cₜₒₜₐₗ as close as possible to C_{total-targeted}
providing the steel sheet with the chemical composition,
annealing the steel sheet according to the selected temperature curve T=Tₙ, as a function of time t, in the selected composition atmosphere.

9. The method as recited in claim 8 wherein C_{T} and C_{L} are calculated through the resolution of the following equations over at least part of the thickness of said steel sheet:
$$\frac{\partial {C}_{L}}{\partial t} + \frac{\partial {C}_{T}}{\partial t} \qquad [ 3 ]$$
$$\frac{\partial {C}_{T}}{\partial t} = \frac{k}{{N}_{L}} {C}_{L} ( {N}_{T} - {N}_{A} \times {C}_{T} ) \qquad [ 4 ]$$
D_{L} being the lattice diffusion coefficient of hydrogen in the crystal lattice,
x being the depth inside said steel sheet,
N_{L} being the volume density of interstitial sites,
N_{T} being the volume density of dislocations,
N_{A} being the Avogadro constant.

10. The method as recited in claim 8 wherein the solubility of the hydrogen C_{H} is calculated through the following equations:
for a temperature T below or equal to Ac3,
$$log ( {C}_{H} ) = 0.5 log ( {p}_{{H}_{2}} ) - 3 - \frac{1500}{{T}_{n}} \qquad [ 1 ]$$
for a temperature T above Ac3,
$$log ( {C}_{H} ) = 0.5 log ( {p}_{{H}_{2}} ) - 2.9 - \frac{1490}{{T}_{n}} \qquad [ 2 ]$$
p_{H2} being the hydrogen partial pressure in the furnace, C_{H} being expressed in at %.

11. The method as recited in claim 8 wherein in the microstructure of the steel sheet includes at least one phase among ferrite, austenite, martensite and bainite and wherein the lattice diffusion coefficient of hydrogen D_{L} is calculated through the following equations
$${D}_{L} = 5.12 * {10}^{- 4} {e}^{- \frac{4150}{{RT}_{n}}}$$
in ferrite, martensite and bainite
$${D}_{L} = 5.8 * {10}^{- 3} {e}^{- \frac{53800}{{RT}_{n}}}$$
in austenite

12. The method as recited in claim 8 wherein the trapping rate of hydrogen k and detrapping rate of hydrogen p are calculated through the following equations
$$k = {k}_{0} exp ( \frac{- {E}_{T}}{{E}_{D}} )$$
$$p = {p}_{0} exp ( \frac{{RT}_{n}}{{RT}_{n}} )$$
with k₀ and p₀ being trapping and detrapping coefficients, E_{T} being the trapping energy, E_{D} being the detrapping energy, and R the universal constant gas.

13. The method as recited in claim 8 wherein volume density of dislocations N_{T} are calculated through the following equation
$${N}_{T} = α \frac{{ρ}_{dis}}{{a}_{bcc} \frac{\sqrt{3}}{2}}$$
ρ_{dis} being the surface density of dislocation, α being the number of dislocations per Burger's vector, and a_{bc} being the lattice parameter.

14. The method as recited in claim 8 wherein the annealing process includes different steps of heating, holding, and cooling the steel sheet.

15. The method as recited in claim 14 wherein the cooling step includes an overaging sub-step, and is followed by a reheating step, followed by a subsequent and final cooling.

16. The method as recited in claim 8 further comprising outputting through a computer display the hydrogen content Cₜₒₜₐₗ at any time to a user.
